(19) United States
(12) Patent Application Publication
(10) Pub. No.: US 2020/0171753 A1
(43) Pub. Date: Jun. 4, 2020

(54) ADDITIVELY-MANUFACTURED GRADIENT GYROID LATTICE STRUCTURES

(71) Applicant: MRL MATERIALS RESOURCES LLC, Beavercreek, OH (US)

(72) Inventors: Daniel P. Satko, Centerville, OH (US); Ayman A. Salem, Beavercreek, OH (US); Thomas Carmody, Dayton, OH (US)

(21) Appl. No.: 16/698,376

(22) Filed: Nov. 27, 2019

Related U.S. Application Data
(60) Provisional application No. 62/773,077, filed on Nov. 29, 2018.
(60) Provisional application No. 62/773,043, filed on Nov. 29, 2018.

(51) Int. Cl. B29C 64/386; B33Y 80/00; B33Y 50/00; A61B 17/86

(52) U.S. Cl. CPC B29C 64/386; A61B 17/863; B33Y 50/00; B33Y 80/00

(57) ABSTRACT

Devices that use additively manufactured connectible unit cells are described in which each unit cell comprises materials and voids. The materials occupy a certain volume of the unit cell with the voids occupying the balance of the volume. The unit cells form a lattice structure, which exhibits smooth transitions between each of the adjacent unit cells. The lattice structure exhibits periodicity along one (1), two (2), or all three (3) dimensions. The materials have a thickness that is a function of the material location within the device.

### CROSS REFERENCE TO RELATED APPLICATIONS

This application claims the benefit of U.S. provisional patent application Ser. No. 62/773,043, filed Nov. 29, 2018, having the title “ADDITIVELY-MANUFACTURED GRADIENT GYROID LATTICE STRUCTURES”, the disclosure of which is hereby incorporated by reference in its entirety, and claims the benefit of U.S. provisional patent application Ser. No. 62/773,077, filed Nov. 29, 2018, having the title “MICROSTRUCTURE-BASED TOPOLOGY OPTIMIZATION FOR STRUCTURAL COMPONENTS MADE BY ADDITIVE MANUFACTURING”, the disclosure of which is hereby incorporated by reference in its entirety.

### STATEMENT REGARDING FEDERALLY SPONSORED RESEARCH OR DEVELOPMENT

This invention was made with government support under N6893618C0012 awarded by the U.S. Department of Defense. The government has certain rights in the invention.

### BACKGROUND

### Field of the Disclosure

The present disclosure relates generally to lattice structures and, more particularly, to additively manufactured gradient gyroid lattice structures.

### Description of Related Art

Typically, hardware is manufactured using homogeneous materials. Because of the homogeneity of the materials, the mechanical properties of the hardware are likewise equally homogeneous. For specialized uses, however, the nearly uniform mechanical properties across the hardware do not provide an optimal solution for that particular use. Consequently, there are ongoing efforts with reference to manufacturing specialized hardware.

### SUMMARY

According to aspects of the present disclosure, devices are provided, which are formed from additively manufactured connectible unit cells in which each unit cell comprises materials and voids. The materials occupy a certain volume of the unit cell with the voids occupying the balance of the volume. Moreover, the unit cells form a lattice structure, which exhibits smooth transitions between each of the adjacent unit cells. In some embodiments, the lattice structure exhibits periodicity along one dimension. In other embodiments, the lattice structure exhibits periodicity along two dimensions. In yet further embodiments the lattice structure exhibits periodicity along three dimensions. The materials have a thickness that is a function of the material location within the device.

According to further aspects of the present disclosure, a three-dimensional (3D) article of manufacture, comprises additively manufactured infinitely connectible triply periodic unit cells, wherein each triply periodic unit cell comprises materials that occupy positions within a portion of a volume of the unit cell. In this regard, the positions are defined by:

0=cos(Pₓᵢ*xᵢ)sin(P_{yi}*yᵢ)+cos(P_{yi}*yᵢ)sin(P_{zi}*zᵢ)+cos(P_{zi}*zᵢ)sin(Pₓᵢ*xᵢ)−T²_{xyzi},

where:
i is an integer that describes an index of an x, y, z coordinate, where each x, y, z coordinate has a single value that satisfies the equation above. The value at each i produces a point cloud described by the equation above;
x is a first dimension;
y is a second dimension;
z is a third dimension;
Pₓ is a periodicity in the x direction;
P_{y} is a periodicity in the y direction;
P_{z} is a periodicity in the z direction; and
T is a thickness.

Each triply periodic unit cell also comprises voids that occupy a balance of the volume of the unit cell. The article of manufacture also comprises a lattice comprising the triply periodic unit cells, wherein the lattice comprises substantially smooth transitions between adjacent triply periodic unit cells, a gradually varying thickness of the materials along x, a gradually varying thickness of the materials along y, and a gradually varying thickness of the materials along z.

According to yet further aspects of the present disclosure, a three-dimensional (3D) article of manufacture, comprises additively manufactured infinitely connectible gyroid unit cells, wherein each gyroid unit cell comprises materials that occupy positions within a portion of a volume of the unit cell. In this regard, the positions are defined by:

0=cos(x)sin(y)+cos(y)sin(z)+cos(z)sin(x), where:
x is a first dimension;
y is a second dimension; and
z is a third dimension.

Each gyroid unit cell also comprises voids that occupy a balance of the volume of the unit cell. The article of manufacture also comprises a lattice comprising the gyroid unit cells, wherein the lattice comprises substantially smooth transitions between adjacent gyroid unit cells, a first periodicity of the materials along x, a second periodicity of the materials along y, a third periodicity of the materials along z, a linearly varying thickness of the materials along x, a gradually varying thickness of the materials along y, and a gradually varying thickness of the materials along z.

According to yet further aspects of the present disclosure, an article of manufacture comprises additively manufactured connectible unit cells. In this regard, each unit cell comprises materials that occupy positions within a volume of the unit cell, and voids that occupy a balance of the volume. The article of manufacture also comprises a lattice comprising the unit cells, where the lattice comprises smooth transitions between adjacent unit cells, a first periodicity of the materials along a first dimension, a second periodicity of the materials along a second dimension, a third periodicity of the materials along a third dimension, and a thickness of the materials as a function of a material location within the article.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a diagram showing one embodiment of an orthopedic screw having an additively manufactured gradient gyroid lattice structure;

FIG. 2 is a diagram showing a close-up view of one embodiment of the additively manufactured gradient gyroid lattice structure within the orthopedic screw of FIG. 1;

FIG. 3 is an illustration of a close-up view of an embodiment of a gyroid equation unit cell;

FIG. 4 is an illustration of a closeup of an embodiment of a gyroid with a 70% void fraction and a 30% solid density; and

FIG. 5 is an illustration of a closeup of an embodiment of a gyroid having a gradient density lattice varying from 100% dense to 10% dense.

### DETAILED DESCRIPTION OF THE EMBODIMENTS

Additive manufacturability of cellular structures with various densities creates challenges due to inherent abrupt transition from solid material to low density cellular structures. These areas of abrupt changes are sources of stress concentrations and could be sources of cracks upon aeroelastic loading. Aspects herein address such areas of abrupt change with a new type of cellular structure with gradient density changes and smooth transitions that result in minimal stress concentration sites, and which can be created using additive manufacturing.

In this regard, aspects herein provide for a gradient Structured Anisotropic Material (SAM), which utilizes cellular structures with different densities in which volumes that does not carry stresses have very low densities and volumes carrying most of the stresses are given relatively higher density, e.g., up to the density of the corresponding solid material.

According to certain aspects herein, for additive manufacturability, a unique unit cell is defined for a scalable cellular structure, namely gyroid unit cell, which is an infinitely connected triply periodic minimal surface. In this regard, the unit cell (gradient gyroid cellular structure) exhibits adaptive densities that can be predicted by SAM.

Thus, aspects of the present disclosure herein provide the ability to create optimized geometries for additively manufactured parts, that leverage knowledge of the underlying microstructure of additive manufacturing material and the role anisotropy plays in supporting external loads.

Hardware that is intended for normal, everyday use is typically manufactured using homogeneous materials that exhibit a relatively homogeneous stress-strain behavior across the hardware. For specialized uses, such as medical or orthopedic applications, it is sometimes beneficial to use inhomogeneous materials that provide non-uniform mechanical properties across the hardware. This is because orthopedic hardware (e.g., nails, pins, screws, plates, etc.) are often load-bearing devices that are affixed internally to bones or other anatomical structures, which tend to apply stresses in many different directions to the orthopedic hardware. Additionally, to facilitate biological growth around the orthopedic hardware, it is sometimes beneficial to have rough or textured surfaces on the orthopedic hardware.

Aspects of the present disclosure using additively manufactured connectible unit cells with materials and voids. The materials occupy a certain volume of the unit cell with the voids occupying the balance of the volume. The unit cells form a lattice structure, which exhibits smooth transitions between each of the adjacent unit cells. The lattice structure exhibits periodicity along one dimension, two dimensions, three dimensions. etc. The materials have a thickness that is a function of the material location within the device.

Having provided a broad technical solution to a technical problem, reference is now made in detail to the description of the embodiments as illustrated in the drawings. While several embodiments are described in connection with these drawings, there is no intent to limit the disclosure to the embodiment or embodiments disclosed herein. On the contrary, the intent is to cover all alternatives, modifications, and equivalents.

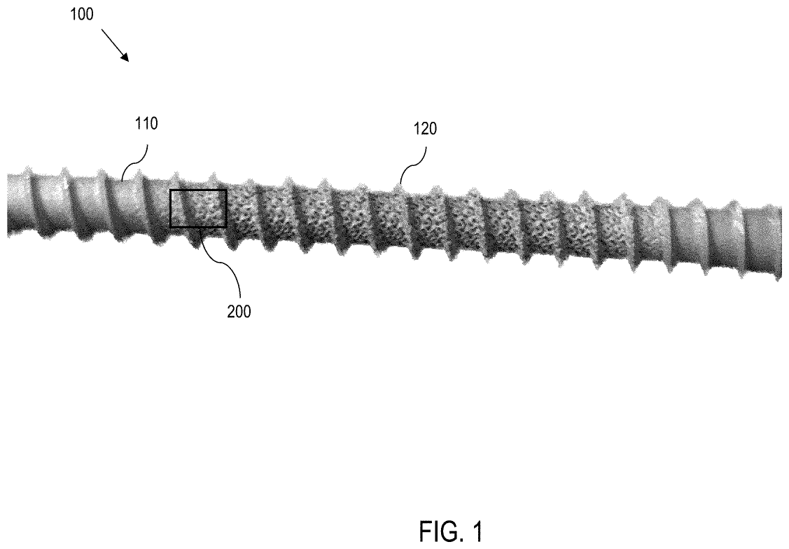

FIG. 1 is a diagram showing one embodiment of a product that takes advantage of the gyroid structures described herein. For sake of discussion, the example embodiment is an orthopedic screw 100. Although a screw 100 is shown as one of many enabling examples, it should be appreciated by those having skill in the art that the following disclosure is applicable to other hardware components, including orthopedic hardware, such as, for example, pins, plates, meshes, clamps, rings, cages, or other implantable rigid structures. Additionally, such structures are not limited to orthopedic hardware but, also, can be extended to other devices, which can include other medical devices, non-medical devices including machine parts, etc.

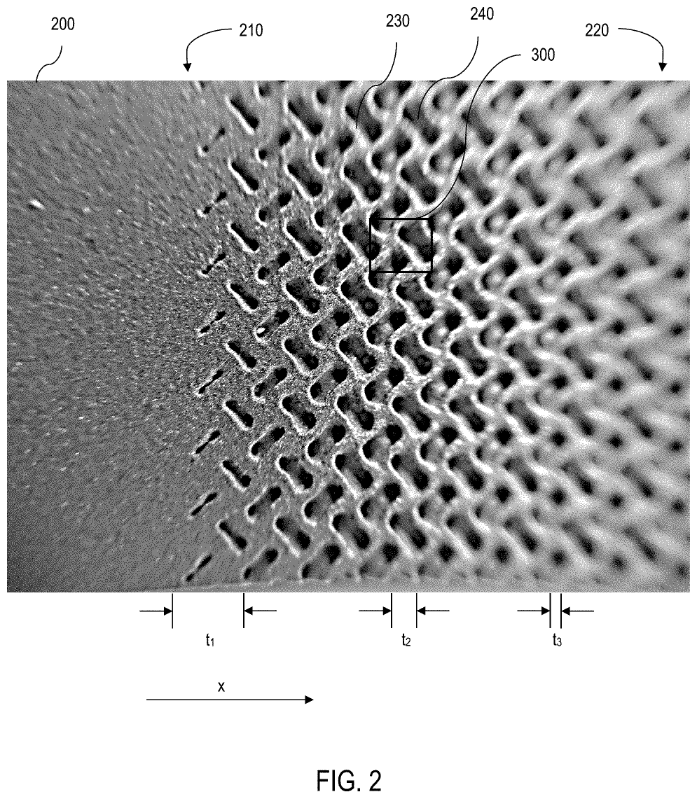

The screw 100 comprises a shaft 110 and threads 120. However, unlike conventional screws, the screw 100 of FIG. 1 comprises an additively manufactured pattern 200 of materials and voids, which is shown in greater detail with reference to FIG. 2. In one embodiment, this pattern 200 is manufactured using topology-optimized additive manufacturing (TOAM) processes to provide for various cellular densities as a function of where the materials and voids are located. TOAM processes typically result in abrupt changes in density, thereby resulting in sources of stress concentrations. However, unlike conventional TOAM processes, as shown in the screw 100 of FIG. 1, a load-bearing portion is designed as a gradient structured anisotropic material (SAM), thereby allowing the gradual density change and smooth transition to alleviate stress-concentration issues.

Turning now to FIG. 2, a close-up view of one embodiment of the pattern 200 of FIG. 1 is shown. In the embodiment of FIG. 2, the pattern comprises an additively manufactured gradient lattice structure 200. As shown in FIG. 2, the gradient progresses along one (1) dimension (x), thereby exhibiting a higher density 210 on one end and a lower density 220 as it progresses along x. The lattice structure 200 comprises unit cells 300, which have materials 230 that occupy certain positions within the volume of the unit cell 300 and voids 240 that occupy the balance of the volume of the unit cell 300.

Because of the gradient along x, the wall thicknesses (t) are thickest (t1) in the −x direction, becoming gradually thinner (t2) and thinner (t3) along the +x direction. In other words, the thickness of the materials (t) is a function of the material location (x) within the lattice 200. Viewed differently, because the unit cells comprise materials 230 and voids 240, the gradient along x can also be considered as a gradual change in density along x. The application of this gradient along x permits smooth transitions between adjacent unit cells 300 along the entire x dimension.

For some embodiments, the density changes from approximately one hundred percent (100%), which implies that the unit cells 300 are all materials 230 and no voids 240, to approximately ten percent (10%), which implies that the unit cells 300 are approximately ninety percent (90%) voids 240 and only approximately ten percent (10%) materials 230. For other embodiments, the density ranges from approximately 100% to approximately twenty percent (20%), while for yet other embodiments, the density ranges from approximately 100% to approximately thirty percent (30%).

In practical applications, the density range of the gradient is dependent on how much stress is exhibited when a load is applied. Thus, for example, when a load is applied to the screw 100 in FIG. 1, the screw 100 exhibits an average stress across the screw 100. Some locations exhibit a higher stress than the average stress, while other locations exhibit lower stress than the average stress. Preferably, the higher-stress locations and lower-stress locations have different densities (meaning, different ratios of material volume to total unit cell volume). As one can appreciate, higher-stress locations will preferably have higher densities (meaning, densities that are higher than the average density), while lower-stress locations will preferably have lower densities (meaning, densities that are lower than the average density).

For some embodiments, the gradient is a linearly varying gradient while, in other embodiments, the gradient is a non-linearly varying gradient. In other words, some embodiments exhibit a linearly varying material thickness as a function of the location along one dimension, while other embodiments exhibit non-linearly varying material thicknesses as a function of the location. Additionally, for some embodiments, the gradient is variable (either linearly or non-linearly) along two (2) dimensions. For yet other embodiments, the gradient is variable (either linearly or non-linearly) along all three (3) dimensions.

Furthermore, in some embodiments, the unit cells 300 are additively manufactured infinitely connectible unit cells that exhibit a periodicity along x. Those having skill in the art will appreciate that lattices are designable with a periodicity in one dimension (1D), a periodicity in two dimensions (2D), or a periodicity in three dimensions (3D). Additionally, depending on the design criteria, the dimensions are cartesian (x, y, z) in some embodiments, while the dimensions are polar (r, θ, z) in other embodiments, and the dimensions are spherical (r, θ, ϕ) in yet other embodiments.

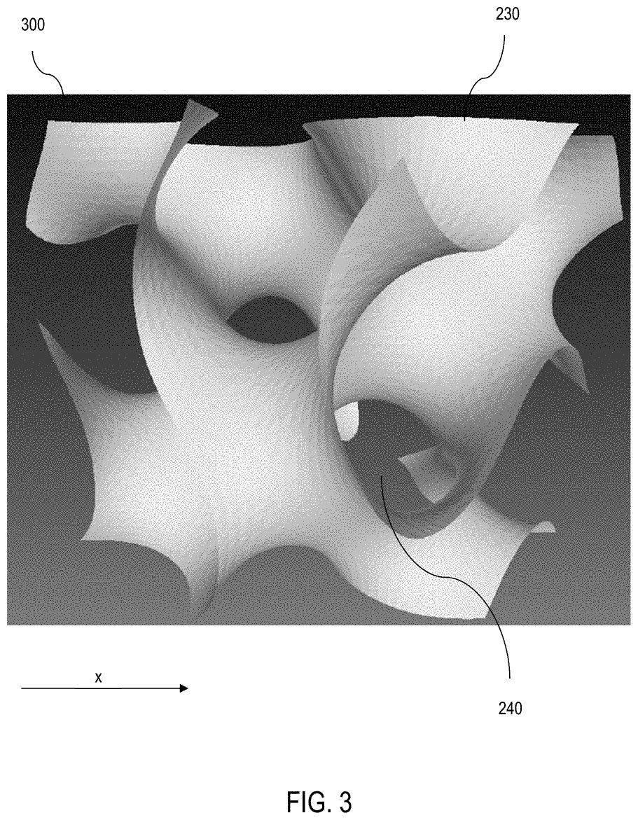

Turning now to FIG. 3, a close-up view of one embodiment of a gyroid unit cell 300 is shown. The illustrated geometry is a single unit cell of the gyroid triply periodic minimal surface.

Specifically, the particular gyroid unit cell 300 of FIG. 3 is described in part, by:

0=cos(x)sin(y)+cos(y)sin(z)+cos(z)sin(x) [Eq. 1],

with (x, y, z) being a position (defined in this embodiment in the cartesian coordinate system) within the unit cell 300 that the materials occupy.

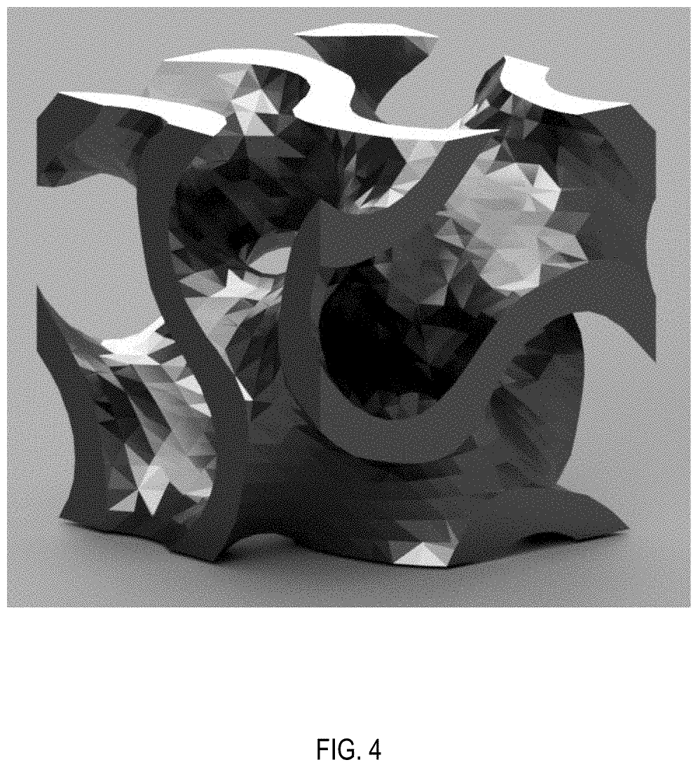

Eq. 1 produces a surface (not a structure with thickness) to produce a gyroid with a thickness, where a volumetric shape with a tailored thickness is built in an additive manufacturing machine, e.g., with the thickness chosen to carry a needed load based upon an underlying design requirement. An example gyroid with a 70% void fraction and a 30% solid density is illustrated in FIG. 4, for purposes of illustration and clarity of discussion herein.

In practical applications, gyroids are defined by a void volume fraction, which can be tailored mathematically and/or experimentally in additive manufacturing, to meet an optimized density based on SAM calculations. Traditionally, a problem exists where high stress concentrations are located at areas with density changes. However, aspects herein provide a Gyroid lattice structure that exhibits a density that changes based on its position. This is referred to herein as “gyroids with gradient density.”

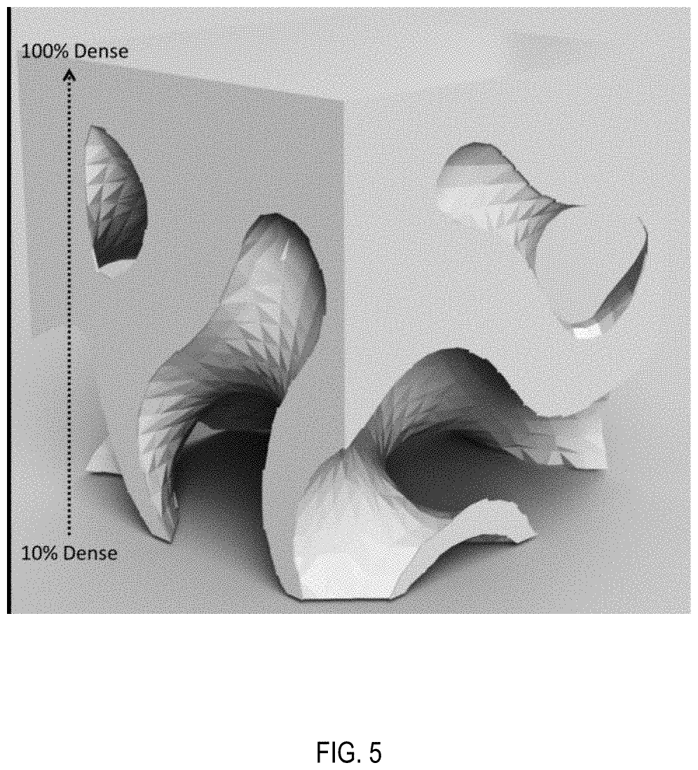

While applicable for linear unidirectional gradients, aspects herein scale to be applied amongst all axes and can follow complex paths in addition to simple linear density changes. In an example implementation, a minimum density is established as 10% in gyroid lattice structures due to discontinuities in the resulting meshes. Referring to FIG. 5, an example illustrates a gyroid with gradient density where the gradient density lattice varies from 100% dense to 10% dense, for purposes of illustration. As illustrated in this example, the structure comprises a top surface attached to a solid material with a 100% density and the bottom volume is at 10% density.

As such, Eq. 1 is provides only the center point of the position. Thus, for a desired density (e.g., 10%, 20%, 30%, 100%, etc.), there is a corresponding material thickness (t) centered on that position. Additionally, while a gradient that varies across unit cells 300 is shown with reference to FIGS. 1 and 2, it should be appreciated that, depending on the design criteria, a gradient that varies within a unit cell 300 is also applicable.

Although a gradient gyroid lattice structure is shown with reference to FIGS. 1 through 3, it should be appreciated that any triply periodic structure (along x, y, and z) also achieves similar benefits as the gyroid lattice structure. Consequently, the gyroid lattice structure is extendable generally to a three-dimensional (3D) article of manufacture, which is additively manufactured using infinitely connectible triply periodic unit cells. Each triply periodic unit cell comprises materials that occupy positions within a portion of a volume of the unit cell. In this regard, the positions are defined by:

0=cos(Pₓᵢ*xᵢ)sin(P_{yi}*yᵢ)+cos(P_{yi}*yᵢ)sin(P_{zi}*zᵢ)+cos(P_{zi}*zᵢ)sin(Pₓᵢ*xᵢ)−T²_{xyzi} [Eq. 2],

where:
i is an integer that describes an index of an x, y, z coordinate, where each x, y, z coordinate has a single value that satisfies Eq. 2 above. The value at each i produces a point cloud described by Eq. 2 above;
x is a first dimension;
y is a second dimension;
z is a third dimension;
Pₓ is 2π times the periodicity in the x direction;
P_{y} is 2π times the periodicity in the y direction;
P_{z} is 2π times the periodicity in the z direction; and
T is a thickness.

Each triply periodic unit cell also comprises voids that occupy a balance of the volume of the unit cell. The article of manufacture also comprises a lattice comprising the triply periodic unit cells, wherein the lattice comprises substantially smooth transitions between adjacent triply periodic unit cells. As one can appreciate, the thickness of the material can be constant along x, linearly varying along x, or gradually varying along x, y, or z, or any combination thereof.

Because cell density is a function of the material-to-void ratio, one way of expressing density (ρ) is as a function of thickness, such as, for example:

ρᵢ=f(Tᵢ) [Eq. 3].

As shown in the FIGURES herein and described above, the use of inhomogeneous materials provides non-uniform mechanical properties. Thus, for orthopedic hardware (e.g., nails, pins, screws, plates, etc.) that often experiences stresses in many different directions, the use of additively manufactured infinitely connectible gradient gyroid unit cells permit custom-tailored hardware that is stronger in higher-stress locations while concurrently using less material in locations that experience lower stresses.

Although exemplary embodiments have been shown and described, a number of changes, modifications, or alterations to the disclosure as described may be made. All such changes, modifications, and alterations should therefore be seen as within the scope of the disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the invention in the form disclosed. Many modifications and variations will be apparent to those of ordinary skill in the art without departing from the scope and spirit of the invention. Aspects of the disclosure were chosen and described in order to best explain the principles of the invention and the practical application, and to enable others of ordinary skill in the art to understand the invention for various embodiments with various modifications as are suited to the particular use contemplated.

What is claimed is:

1. A three-dimensional (3D) article of manufacture, comprising:
additively manufactured infinitely connectible gyroid unit cells, wherein each gyroid unit cell consists of:
materials that occupy positions within a portion of a volume of the unit cell, wherein the positions are defined by:
0=cos(x)sin(y)+cos(y)sin(z)+cos(z)sin(x), wherein:
x is a first dimension;
y is a second dimension; and
z is a third dimension; and
voids that occupy a balance of the volume of the unit cell;
a lattice comprising the gyroid unit cells, wherein the lattice comprises:
substantially smooth transitions between adjacent gyroid unit cells;
a first periodicity of the materials along x;
a second periodicity of the materials along y;
a third periodicity of the materials along z;
a linearly varying thickness of the materials along x;
a gradually varying thickness of the materials along y; and
a gradually varying thickness of the materials along z.

2. An article of manufacture, comprising:
additively manufactured connectible unit cells, wherein each unit cell comprises:
materials that occupy positions within a volume of the unit cell; and
voids that occupy a balance of the volume;
a lattice comprising the unit cells, wherein the lattice comprises:
smooth transitions between adjacent unit cells;
a first periodicity of the materials along a first dimension;
a second periodicity of the materials along a second dimension;
a third periodicity of the materials along a third dimension; and
a thickness of the materials as a function of a material location within the article.

3. The article of manufacture of claim 2, wherein the positions that the materials occupy are defined by:
0=cos(x)sin(y)+cos(y)sin(z)+cos(z)sin(x), wherein:
x is a first dimension;
y is a second dimension; and
z is a third dimension.

4. The article of manufacture of claim 2, wherein the infinitely connectible unit cells comprise gyroid unit cells.

5. The article of manufacture of claim 2, wherein the thickness of the materials varies linearly as a function of its location along the first dimension.

6. The article of manufacture of claim 2, wherein the thickness of the materials varies linearly as a function of its location along the second dimension.

7. The article of manufacture of claim 2, wherein the thickness of the materials varies linearly as a function of its location along the third dimension.

8. The article of manufacture of claim 2, wherein the thickness of the materials varies linearly as a function of its location along at least two (2) of the three (3) dimensions.

9. The article of manufacture of claim 2, wherein the thickness of the materials varies linearly as a function of its location along all the three (3) dimensions.

10. The article of manufacture of claim 2, wherein the thickness of the materials varies non-linearly as a function of its location along the first dimension.

11. The article of manufacture of claim 2, wherein the thickness of the materials varies non-linearly as a function of its location along the second dimension.

12. The article of manufacture of claim 2, wherein the thickness of the materials varies non-linearly as a function of its location along the third dimension.

13. The article of manufacture of claim 2, wherein the thickness of the materials varies non-linearly as a function of its location along at least two (2) of the three (3) dimensions.

14. The article of manufacture of claim 2, wherein the thickness of the materials varies non-linearly as a function of its location along all the three (3) dimensions.

15. The article of manufacture of claim 2, further comprising a density that is calculated as a ratio of a volume of the materials within the unit cell to the volume of the unit cell, wherein the density is a function of the material location, wherein the density is between approximately ten percent (10%) and approximately one hundred percent (100%).

16. The article of manufacture of claim 15, wherein the density varies linearly along one (1) of the three (3) dimensions.

17. The article of manufacture of claim 15, wherein the density varies linearly along two (2) of the three (3) dimensions.

18. The article of manufacture of claim 15, wherein the density varies linearly along all three (3) dimensions.

19. The article of manufacture of claim 15, wherein the density varies non-linearly along one (1) of the three (3) dimensions.

20. The article of manufacture of claim 15, wherein the density varies non-linearly along two (2) of the three (3) dimensions.

21. The article of manufacture of claim 15, wherein the density varies non-linearly along all three (3) dimensions.

22. The article of manufacture of claim 2, further comprising a density that is calculated as a ratio of a volume of the materials within the unit cell to the volume of the unit cell, wherein the density is a function of the material location, wherein the density is between approximately twenty percent (20%) and approximately one hundred percent (100%).

23. The article of manufacture of claim 2, further comprising a density that is calculated as a ratio of a volume of the materials within the unit cell to the volume of the unit cell, wherein the density is a function of the material location, wherein the density is between approximately thirty percent (30%) and approximately one hundred percent (100%).

24. The article of manufacture of claim 2, wherein the article of manufacture exhibits an average stress across the article when a load is applied to the article, wherein the material location comprises:
higher-stress locations exhibiting stresses that are higher than the average stress; and
lower-stress locations exhibiting stress that are lower than the average stress.

25. The article of manufacture of claim 24, further comprising densities calculated as a ratio of a volume of the materials in the unit cell to a total volume of the unit cell, wherein the densities comprise:
an average density;
higher densities that are higher than the average density; and
lower densities that are lower than the average density.

26. The article of manufacture of claim 25, wherein:
the higher-stress locations exhibit higher densities; and
the lower-stress locations exhibit lower densities.

27. The article of manufacture of claim 2, wherein the article of manufacture comprises a medical device that exhibits an average stress across the device when a load is applied to the medical device, wherein the medical device comprises an average density that is a ratio of an average volume of the materials within the unit cell to a total volume of the unit cell, wherein the medical device further comprises:
higher-stress locations exhibiting stresses that are higher than the average stress, wherein the higher-stress locations have higher densities, wherein the higher densities are higher than the average density; and
lower-stress locations exhibiting stresses that are lower than the average stress, wherein the lower-stress locations have lower densities, wherein the lower densities are lower than the average density.

28. The article of manufacture of claim 27, wherein the medical device is orthopedic hardware.

29. The article of manufacture of claim 27, wherein the medical device is one selected from the group consisting of:
a pin;
a screw;
a plate;
a mesh;
a clamp;
a ring;
a cage; and
any other implantable rigid structure.

30. The article of manufacture of claim 2, wherein the first dimension (x), the second dimension (y), and the third dimension (z) are dimensions in a cartesian coordinate system.

31. The article of manufacture of claim 2, wherein the first dimension (r), the second dimension (θ), and the third dimension (z) are dimensions in a polar coordinate system.

32. The article of manufacture of claim 2, wherein the first dimension (r), the second dimension (θ), and the third dimension (ϕ) are dimensions in a spherical coordinate system.

33. The article of manufacture of claim 2, wherein the positions that the materials occupy are defined by an equation:
0=cos(Pₓᵢ*xᵢ)sin(P_{yi}*yᵢ)+cos(P_{yi}*yᵢ)sin(P_{zi}*zᵢ)+cos(P_{zi}*zᵢ)sin(Pₓᵢ*xᵢ)−T²_{xyzi},
wherein:
i is an integer that describes an index of an x, y, z coordinate, where each x, y, z coordinate has a single value that satisfies the equation;
x is a first dimension;
y is a second dimension;
z is a third dimension;
Pₓ is a periodicity in the x direction;
P_{y} is a periodicity in the y direction;
P_{z} is a periodicity in the z direction; and
T is a thickness.
